(19) United States
(12) Patent Application Publication
(10) Pub. No.: US 2003/0188749 A1
(43) Pub. Date: Oct. 9, 2003

(54) Systems and methods for endotracheal intubation

(76) Inventors: Travis R. Nichols, Perry, UT (US); David Hoffman, Lovell, WY (US); Ken Ferbrache, Lovell, WY (US)

Correspondence Address: KIRTON AND MCCONKIE, 1800 EAGLE GATE TOWER, 60 EAST SOUTH TEMPLE, P O BOX 45120, SALT LAKE CITY, UT, 84145-0120, US

(21) Appl. No.: 10/116,895

(22) Filed: Apr. 5, 2002

(51) Int. Cl. A61M 16/00

(52) U.S. Cl. 128/207.14

(57) ABSTRACT

Systems and methods for using a guidewire to intubate a standard endotracheal tube in a patient. An insertion mechanism is used to enable delivery of a guidewire to the tracheal cavity of a patient. The guidewire is then threaded up the tracheal cavity, through the glottic opening and up either the oropharyngeal airway, so as to extend out of the patient's mouth, or up the nasopharyngeal airway, so as to extend out of the patient's a nostril. A typical endotracheal tube, having a channel that extends from a proximal end to a distal end of the tube, is coupled to the guidewire and is positioned into the tracheal cavity of the patient by using the guidewire to withdraw the tube down the airway and into the trachea. The endotracheal tube is decoupled from the guidewire, and the guidewire is removed from the patient, leaving the endotracheal tube properly intubated in the patient.

### BACKGROUND OF THE INVENTION

1. Field of the Invention

The present invention relates to endotracheal intubation. More particularly, the present invention relates to systems and methods for facilitating the intubation of a standard endotracheal tube in a patient and for ensuring proper placement of the tube.

2. Background and Related Art

At times it is advantageous to position an endotracheal tube in an airway of a patient. One positioning of an endotracheal tube extends from the patent's mouth, down his/her oropharyngeal airway, and down at least a portion of his/her trachea. Another positioning of an endotracheal tube extends from the patient's nose, down his/her nasopharyngeal airway, and down at least a portion of his/her trachea. The placement of an endotracheal tube isolates the airway of the patient, reduces the risk of aspiration, permits suctioning of the trachea, ensures delivery of a high concentration of oxygen, provides a route for the administration of particular drugs, and ensures delivery of a selected tidal volume to maintain adequate lung inflation.

Current medical procedures available for performing the intubation require the advanced skill of a highly trained practitioner due to the risk of complications to the patient. During endotracheal intubation, the maximum interruption of the patient's ventilation is approximately 30 seconds, and preferably 15 seconds. Adequate ventilation and oxygenation must be provided to the patient between intubation attempts. Typically, a second practitioner is used during intubation to provide cricoid pressure to the patient in order to ensure proper placement of the tube in the patient's trachea.

Equipment used to perform an intubation procedure includes a laryngoscope having a power source and a light source used to expose the glottis, an endotracheal tube that is open at both ends, a malleable stylet that may be inserted down the tube to enable the tube to conform to any desired configuration so as to facilitate the insertion of the tube into the larynx and trachea, a syringe for cuff inflation, magill forceps for removing foreign material or for directing the tip of the tube into the larynx, a water-soluble lubricant, and a suction unit. Before insertion, the tube may be lubricated with the water-soluble lubricant and the patient's head is properly positioned. A practitioner opens the patient's mouth and places the blade of the laryngoscope in the right side of the mouth, displacing the patient's tongue to the left. Gentleness and the avoidance of pressure on the patient's lips and teeth are essential. The endotracheal tube is advanced through the right corner of the mouth and, under direct vision, through the vocal cords. The stylet is removed from the tube.

Tube placement is confirmed simultaneously with delivery of the first manual breath by auscultating the epigastrium while observing the chest wall for evidence of thoracic inflation. If stomach gurgling occurs and chest wall expansion is not evident, inadvertent esophageal intubation is presumed and no further breaths are delivered. In such a situation, proper placement of the tube is reattempted after the patient has been well oxygenated. At times a chest x-ray is obtained to confirm proper placement of the tube.

While current techniques enable placement of an endotracheal tube in an airway of a patient, the techniques require an advanced level of skill and may result in trauma to the patient. The patient's lips or tongue may be compressed and lacerated between the blade of the laryngoscope and the teeth. The teeth may be chipped. The tip of the tube or the stylet may lacerate the pharyngeal or tracheal mucosa, resulting in bleeding, hemotoma, or abscess formation. Rupture of the trachea and damage to the vocal cords may also occur. Insertion of the endotracheal tube into a main bronchus may result in hypoxemia due to under-inflation of the other lung. Similarly, accidental insertion of the endotracheal tube into the esophagus may result in no ventilation or oxygenation. Accordingly, it would be an advancement in the art to improve or replace current techniques relating to endotracheal intubation.

### SUMMARY OF THE INVENTION

The present invention relates to endotracheal intubation. More particularly, the present invention relates to systems and methods for facilitating the intubation of an endotracheal tube in a patient and for ensuring proper placement of the tube.

Implementation of the present invention takes place in association with a guidewire that is configured to properly position an endotracheal tube. In one implementation, the endotracheal tube has a proximal end, a distal end, and a channel that is defined by an inside wall extending from the proximal end to the distal end of the tube and allows air to flow therethrough upon intubation in the patient. A distal end of an insertion mechanism may be used to insert the guidewire through a front portion of the patient's neck and into the trachea. In a further implementation, the insertion mechanism includes a catheter needle and a sheath, having a channel and a head. Once inserted, the needle is withdrawn, leaving the sheath inserted in the patient. The head of the sheath is located at the outside surface of the patient's neck, and the channel is configured to receive the guidewire for delivery to the patient's trachea. Once the sheath is positioned so as to extend to the inside of the trachea, the guidewire is threaded through the channel, up the tracheal cavity toward the head, through the glottic opening and up either the oropharyngeal airway, so as to extend out the mouth, or up the nasopharyngeal airway, so as to extend out a nostril.

While extending the guidewire out from either a nostril or the mouth, the endotracheal tube is threaded onto the guidewire and optionally coupled to the guidewire. In one implementation, the endotracheal tube is coupled to the guidewire by the use of a clamp placed at a distal portion of the guidewire. In a further implementation, the guidewire includes or is associated with a coupling mechanism that couples at least a portion of the guidewire with at least a portion of a standard endotracheal tube. An example of a coupling mechanism includes a bulb or cuff that is selectively inflated to couple the outer surface of the bulb or cuff with a portion of an inner wall of the tube. Other examples of a coupling mechanism include a clamp, a fastener, a tie, or any other device that couples the guidewire to the endotracheal tube. In a further embodiment, a coupling mechanism includes a plurality of coupling devices, such as an inflatable bulb and a clamp.

Once coupled, the proximal end of the endotracheal tube is properly positioned into the tracheal cavity of the patient by using the guidewire to withdraw the proximal end of the tube down the oropharyngeal or nasopharyngeal airway, past the glottic opening and down the trachea. The guidewire may be pulled at the proximal end and/or the guidewire and/or tube may be pushed at the distal end. Once the proximal end of the tube is located in the tracheal cavity, the guidewire is decoupled from the tube and removed from the patient, leaving the endotracheal tube properly positioned within the patient.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

### BRIEF DESCRIPTION OF THE DRAWINGS

In order that the manner in which the above recited and other features and advantages of the present invention are obtained, a more particular description of the invention will be rendered by reference to specific embodiments thereof, which are illustrated in the appended drawings. Understanding that the drawings depict only typical embodiments of the present invention and are not, therefore, to be considered as limiting the scope of the invention, the present invention will be described and explained with additional specificity and detail through the use of the accompanying drawings in which:

FIG. 1 illustrates a perspective view of a representative endotracheal intubation system that may be used in accordance with the present invention;

FIG. 2 illustrates a representative embodiment of a channel configured to receive a guidewire for endotracheal intubation;

FIG. 3 illustrates the utilization of a representative insertion mechanism into a patient;

FIG. 4 illustrates the threading of a guidewire up the trachea and out the oropharyngeal airway, and the coupling of the guidewire to an endotracheal tube;

FIG. 5 illustrates utilizing the guidewire to properly position the distal end of the endotracheal tube in the tracheal cavity; and

FIG. 6 illustrates a representative embodiment for providing endotracheal intubation in accordance with the present invention.

### DETAILED DESCRIPTION OF THE INVENTION

The present invention relates to endotracheal intubation. More particularly, the present invention relates to systems and methods for facilitating the intubation of an endotracheal tube in a patient and for ensuring proper placement of the tube.

In the disclosure and in the claims the term “standard endotracheal tube” shall refer to any endotracheal tube that may be intubated into a patient to facilitate breathing, and which is not specifically designed to be associated with a particular a guidewire. Thus, in accordance with embodiments of the present invention, a guidewire is provided to associate with any type of endotracheal tube available to facilitate endotracheal intubation.

In the disclosure and in the claims the term “insertion mechanism” shall refer to any device, process or procedure that may be used to insert a guidewire into the tracheal cavity of a patient. One example of an insertion mechanism includes a needle, such as a catheter needle, having a channel extending therethrough that is configured to receive a guidewire and deliver the guidewire to the tracheal cavity of the patient. Another example of an insertion mechanism includes a sheath having a channel that is configured to receive a guidewire to intubate a patient, as will be further discussed below. Those skilled in the art shall appreciate that the term insertion mechanism embraces other devices, processes, and/or procedures that may be used to allow a guidewire to be used in accordance with the present invention to intubate an endotracheal tube in a patient.

Embodiments of the present invention take place in association with a guidewire that is configured to position a typical endotracheal tube, which includes a channel that extends from a proximal end of the tube to a distal end of the tube and allows air to flow upon intubation of the tube in an airway of the patient. In one embodiment, at least a portion of the guidewire includes a coating, such as a coating that includes a lubricating material or another material that facilitates threading the guidewire through a patient.

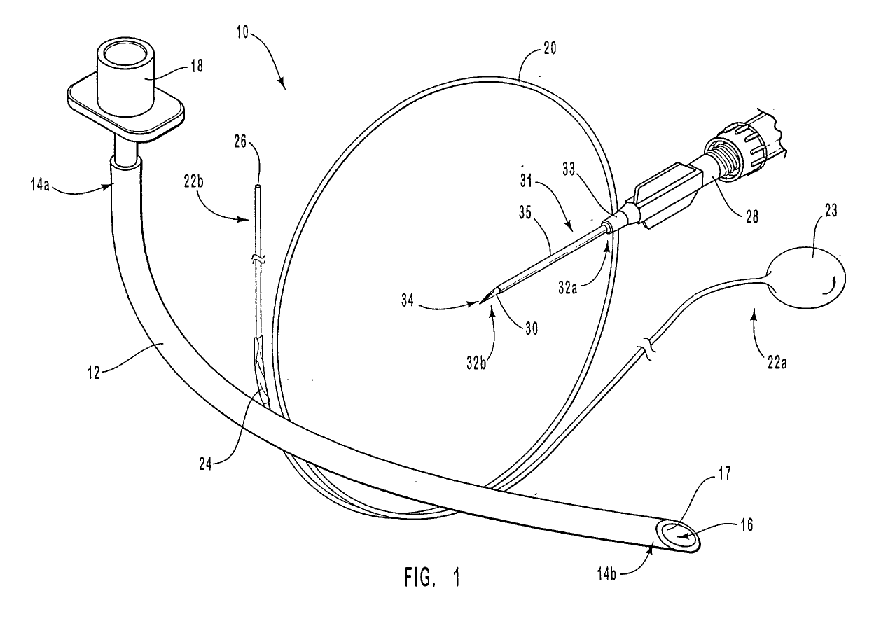

While FIG. 1 and the corresponding discussion are intended to provide a general description of a representative system that may be used in accordance with the present invention to perform an endotracheal intubation procedure on a patient, embodiments of the present invention may be practiced with any configuration of an endotracheal tube that includes a channel extending therethrough. Furthermore, embodiments of the present invention embrace the use of endotracheal tubes that are configured to be used in either the oropharyngeal airway or in the nasopharygeal airway of a patient.

With reference to FIG. 1, a perspective view of a representative system 10 is illustrated, and includes an endotracheal tube 12, a guidewire 20 and an insertion mechanism, such as insertion mechanism 28. The endotracheal tube 12 comprises a flexible material to facilitate intubation and includes a proximal end 14a and a distal end 14b. A channel 16, which is defined by an inside wall 17 of tube 12, extends from the proximal end 14a to the distal end 14b of the tube 12. In one embodiment, a hub 18 is configured to be coupled to the proximal end 14a of the tube 12. The hub enables coupling of the tube 12 to a medical device, such as a respirator or other device.

Guidewire 20 comprises a dynamic material, which may be coated by a lubricating material, such as a polymer or other material, and includes a proximal end 22a and a distal end 22b. The distal end 22b is delivered to the tracheal cavity of a patient and directed up either the patient's oropharyngeal or nasopharygeal airway to properly intubate an endotracheal tube 12 into a patient, as will be discussed below. As such, the distal end 22b of guidewire 20 includes a blunt end 26 to enable threading of the guidewire 20 up an airway of the patient.

In the illustrated embodiment, guidewire 20 includes a bulb or cuff, illustrated as bulb 24, which may be selectively inflated. In one embodiment, the bulb 24 comprises a dynamic material, such as a polymer, that is capable of withstanding the forces exerted thereon when the bulb 24 is inflated. An inflation device, such as pump 23, is coupled to guidewire 20 to selectively deliver or remove air from bulb 24 to respectively inflate or deflate bulb 24. Bulb 24 is deflated to enable delivery of the guidewire 20 to the tracheal cavity of a patient and to direct distal end 22b of guidewire 20 up either the oropharyngeal or nasopharygeal airway of a patient. Bulb 24 may be inflated to couple endotracheal tube 12 to guidewire 20. Further, bulb 24 may be deflated to decouple guidewire 20 from endotracheal tube 12 in order to remove guidewire 20 from the patient once the endotracheal tube 12 is properly positioned.

While FIG. 1 illustrates the use of a pump 23, those skilled in the art will appreciate that a variety of electrical and/or mechanical inflation devices may be used to selectively inflate bulb 24. Alternatively or additionally, a clamp or other similar attachment device may be used to couple tube 12 to guidewire 20 in order to perform an endotracheal intubation procedure in accordance with the present invention.

An insertion mechanism is used to selectively deliver guidewire 20 to the tracheal cavity of a patient. One example of an insertion mechanism is catheter 28, which includes needle 30, having proximal end 32a and distal end 32b, and sheath 31. The distal end 32b of needle 30 is configured to facilitate the insertion of needle 30 and sheath 31 into a patient. Sheath 31 includes head 33 and channel 35. The length of needle 30 and sheath 31 enables a distal portion of channel 35 to be located in the tracheal cavity when needle 30 is inserted into a patient.

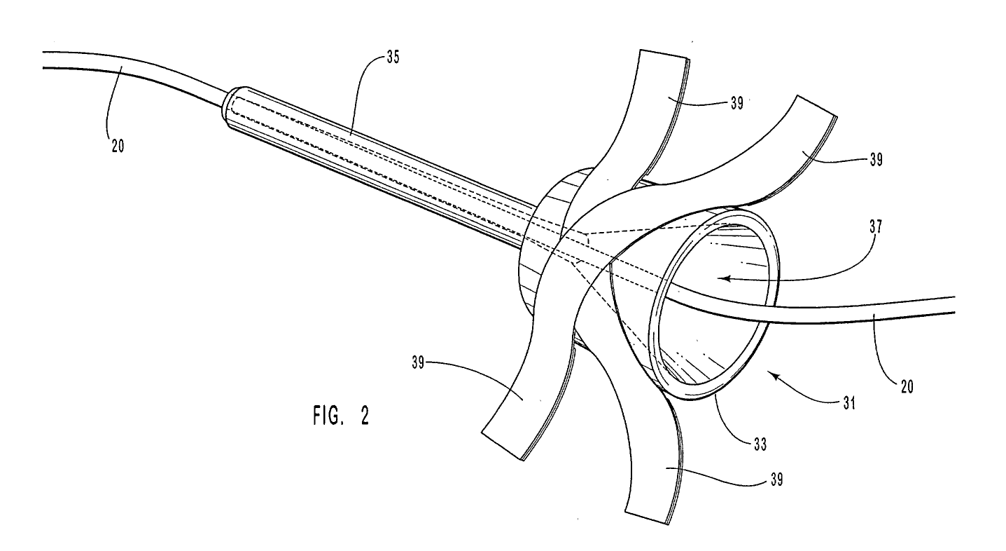

With reference now to FIG. 2, a representative embodiment of sheath 31 is provided with additional detail. In FIG. 2, sheath 31 includes head 33 and channel 35. In the illustrated embodiment, the inside wall 37 of head 33 is in a conical shape to configure head 31 to receive guidewire 20. Once received, guidewire 20 is threaded through channel 35 and delivered into the tracheal cavity of the patient. In one embodiment, the delivery location of the guidewire is the location where the proximal end of the endotracheal tube is to be located in the tracheal cavity, as will be demonstrated below.

The diameter of channel 35 is slightly larger than the diameter of guidewire 20 to enable guidewire 20 to move within channel 35 in order for guidewire 20 to be directed up either the oropharyngeal or nasopharygeal airway of a patient. One or more couplers 39 (e.g., strips of adhesive tape, or other means for coupling sheath 31 to a patient) may be used to retain sheath 31 in the patient.

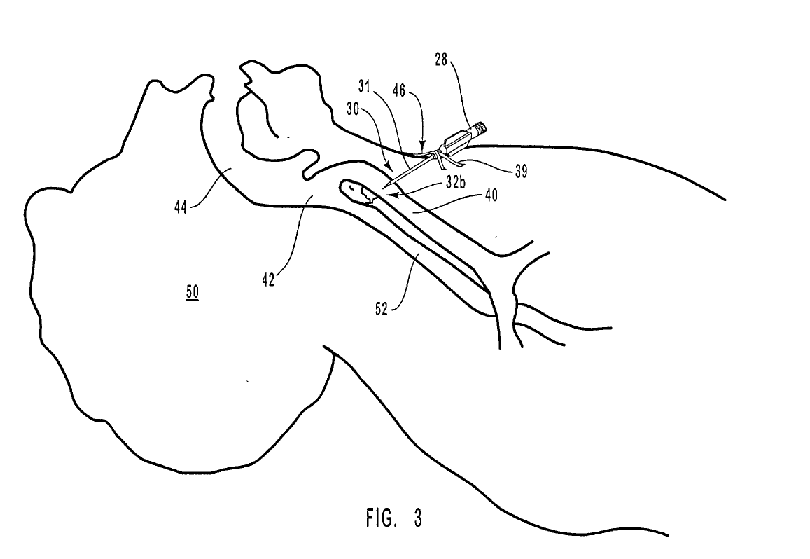

With reference now to FIGS. 3-5, a representative example is provided for the utilization of a guidewire to properly intubate an endotracheal tube into an airway of a patient in accordance with the present invention. In FIG. 3, a patient 50 is provided having a tracheal cavity 40, a glottic opening 42, an oropharyngeal airway 44 and an esophagus 52. A distal end 32b of an insertion mechanism is inserted through a front portion 46 of the neck of patient 50 and into the tracheal cavity 40. In the illustrated embodiment, the insertion mechanism includes needle 30 and sheath 31. The insertion mechanism is inserted into patient 50 so that a distal end of sheath 31 extends into tracheal cavity 40. Once inserted, sheath 31 is affixed to patient 50 by attaching couplers 39 onto the exterior surface of the patient's neck. Catheter 28 is then removed from patient 50, leaving sheath 31 installed in patient 50 for ingress of a guidewire into the tracheal cavity 40 of the patient 50.

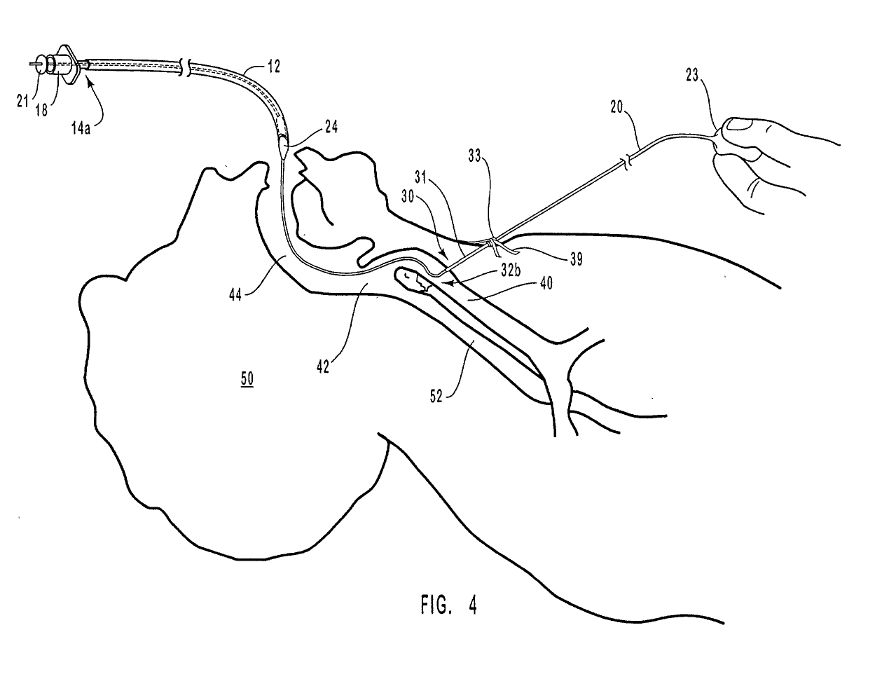

With reference now to FIG. 4, once sheath 31 is properly positioned and secured so as to extend to the inside of the tracheal cavity 40, guidewire 20 is threaded through channel 31 of sheath 31, up the tracheal cavity 40 toward the head, through the glottic opening 42 and up the oropharyngeal airway 44 so as to extend distal end 22b out of the mouth 48 of patient 50. In FIG. 4, an endotracheal tube 12 is provided, which includes a channel that is defined by an inside wall 17 (FIG. 1) of the tube 12 and that extends from the proximal end 14a (FIG. 1) to the distal end 14b of the tube 12. Endotracheal tube 12 optionally includes hub 18 for attachment onto a medical device, such as a respirator. In accordance with embodiments of the present invention, once guidewire 20 is threaded through an airway of patient 50 and through tube 12, endotracheal tube 12 may be coupled to guidewire 20.

In one embodiment, a clamp or other coupler, illustrated as clamp 21, is employed to couple tube 12 to guidewire 20. As such, since guidewire 20 is located in the patient's airway, guidewire 20 enables the proper positioning or intubation of tube 12 in the patient's airway.

Additionally or alternatively, guidewire 20 includes a cuff or bulb, illustrated as bulb 24, which is selectively inflated by using a pump 23. In one embodiment, the bulb 24 is positioned and inflated outside of the patient's mouth such that a distal portion of bulb 24 is in contact with a proximal portion of the inside wall of tube 12 and a proximal portion of bulb 24 is outside of and abutting against the proximal end of tube 12. As such, bulb 24 assists in the coupling of tube 12 to guidewire 20 and facilitates the threading of tube 12 into the patient and down the tracheal cavity 40, reducing any catching of the proximal end of tube 12 with internal tissue of the patient 50. In other words, the inflated bulb provides a cone or taper on the proximal end of the tube 12 to move soft tissue (e.g., vocal chords, etc.) out and around the proximal end of the tube 12.

In a further embodiment, bulb 23 may be used as a handle to withdraw guidewire 20 from patient 50. In other embodiments, bulb 24 includes a configuration, such as a rib configuration, a doughnut configuration, or another configuration, which allows air to flow and pass through a channel of the endotracheal tube while the bulb is inflated. Similarly, in one embodiment, clamp 21 is configured to allow air to flow/pass while coupling tube 12 to guidewire 20.

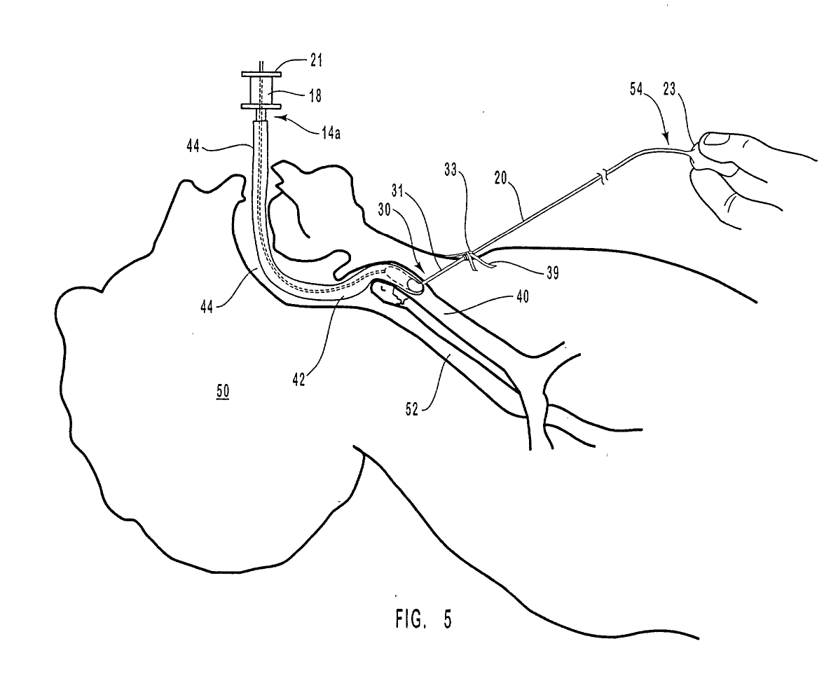

Thus, once tube 12 is coupled to guidewire 20, endotracheal tube 12 is properly positioned into the tracheal cavity 40 of the patient 50 by withdrawing the guidewire 20 down the oropharyngeal airway 44, past the glottic opening 42 and down the tracheal cavity 40. And, since guidewire 20 is coupled to endotracheal tube 12, the withdrawal of guidewire 20 brings endotracheal tube 12 therewith. As such, in accordance with the present invention, a guidewire may be used to guide endotracheal tube 12 to a proper placement within the tracheal cavity 40 of patient 50. The positioning of tube 12 into the tracheal cavity 40 is illustrated in FIG. 5.

Once the distal end of endotracheal tube 12 is properly located in the tracheal cavity 40 of patient 50, the guidewire 20 is decoupled from the tube 12. Thus, when bulb 24 is used, the decoupling includes deflating bulb 24. Further, clamp 21 may or may not require the removal from guidewire 20 to decouple tube 12 from guidewire 20. Further, once decoupled, embodiments of the present invention embrace the removal of guidewire 20 from either the channel created by sheath 31 or from out of the mouth.

In one embodiment, where guidewire 20 is removed from a patient 50 through the mouth, pump 23 is decoupled from guidewire 20 at a location between pump 23 and the neck of patient 50, such as at location 54. In one embodiment, the decoupling of pump 23 occurs by cutting guidewire 20 at the location. Once guidewire 20 is decoupled from tube 12, a distal portion of guidewire 20 may be pulled at or near the patient's mouth and/or a proximal end of guidewire 20 may be pushed to remove guidewire 20 from the patient. Thus, in accordance with the process illustrated in FIGS. 3-5, the intubation of an endotracheal tube in a patient is facilitated and proper placement of the tube into a tracheal cavity is ensured.

While the example provided in FIGS. 3-5 and the corresponding disclosure references endotracheal intubation down the oropharyngeal airway of a patient, embodiments of the present invention also embrace endotracheal intubation down the nasopharyngeal airway. This is performed by delivering a guidewire to the tracheal cavity of a patient and threading the guidewire up the tracheal cavity toward the head, through the glottic opening, and up the nasopharyngeal airway of the patient so as to extend a distal end of the guidewire out of a nostril of patient. Once the guidewire is positioned so as to extend from a nostril, an endotracheal tube is intubated into a patient by using the threaded guidewire.

In one embodiment, the endotracheal tube is coupled to the guidewire, such as through the use of a clamp, a bulb, and/or another coupling device. The endotracheal tube is then properly positioned into the tracheal cavity of the patient by withdrawing the guidewire down the nasopharyngeal airway and down the tracheal cavity. And, since the guidewire is coupled to the endotracheal tube, the withdrawal of the guidewire brings the endotracheal tube therewith. The tube is then decoupled from the guidewire, and the guidewire is removed from the patient.

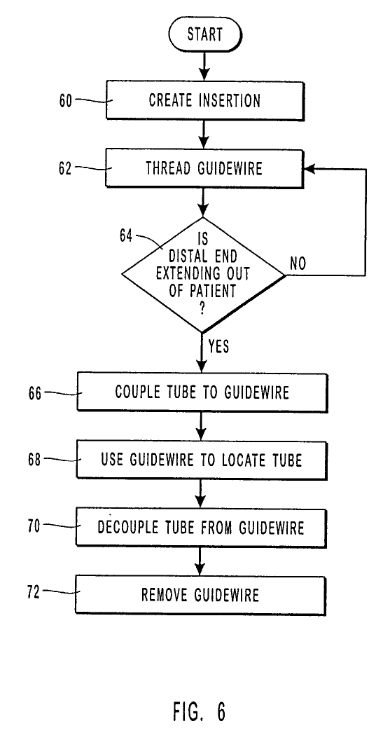

With reference now to FIG. 6, a representative method is provided for endotracheal intubation in accordance with the present invention. In FIG. 6, execution begins at step 60, where an insertion mechanism comprising a needle, a sheath, and/or another device creates an insertion into the patient to deliver at least a portion of a guidewire to the tracheal cavity of the patient. At step 62, the guidewire is threaded up the tracheal cavity toward the head of the patient, through the glottic opening and up either the oropharyngeal airway or up the nasopharyngeal airway.

At decision block 64 a determination is made as to whether or not the distal end of the guidewire is extending out of the patient. For example, if the guidewire was threaded up the oropharyngeal airway, the guidewire will extend out of the patient's mouth. Alternatively, if the guidewire was threaded up the nasopharyngeal airway, the guidewire will extend out of a nostril of the patient. Therefore, if it is determined at decision block 64 that the distal end of the guidewire is not extending out of the patient, execution returns back to 62 in order to allow the guidewire to be threaded such that a portion of the distal end of the guidewire extends from the patient.

When it is determined at decision block 64 that the distal end of the guidewire is extending from the patient, an endotracheal tube is coupled to the guidewire. At step 68 the guidewire is used to properly locate the endotracheal tube into the tracheal cavity of the patient. Withdrawing the guidewire down the patient's airway and into the tracheal cavity performs this placement since the guidewire and the tube are coupled.

At step 70 the tube is decoupled from the guidewire. In one embodiment, the decoupling includes deflating the bulb and may include the removal of one or more clamps. Once decoupled, execution then proceeds to step 72, where the guidewire is removed from the patient. In one embodiment, the removal includes cutting the guidewire to remove a pump from the guidewire and pulling the guidewire for removal of the guidewire from the patient's mouth.

Thus, as discussed herein, embodiments of the present invention embrace the utilization of a guidewire to position or intubate an endotracheal tube in a patient. The guidewire is threaded up an airway of the patient and a distal portion of the guidewire may be coupled to an endotracheal tube, using for example a clamp and/or an inflatable cuff. Then, since the guidewire was previously threaded up the airway, the guidewire is used to properly position the endotracheal tube in the patient's airway. The guidewire is then removed, leaving the properly intubated tube in the patient.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

What is claimed is:

1. An intubation system configured to facilitate the positioning of an endotracheal tube in a patient, the system comprising:
a guidewire; and
a coupling mechanism to selectively couple the guidewire to a standard endotracheal tube, wherein the coupling mechanism comprises at least one of:
(i) a clamp; and
(ii) an inflatable bulb.

2. An intubation system as recited in claim 1, wherein the inflatable bulb is coupled to the guidewire and to a pump, which selectively inflates the bulb to couple the guidewire to a portion of an inside wall of the endotracheal tube.

3. An intubation system as recited in claim 2, wherein the inflated bulb provides a taper on a proximal end of the tube.

4. An intubation system as recited in claim 2, wherein the inflated bulb includes a configuration that allows air to pass through the channel while the bulb is inflated.

5. An intubation system as recited in claim 1, wherein at least a portion of the guidewire includes a coating thereon, wherein the coating includes a lubricating material.

6. An intubation system as recited in claim 1, further comprising an insertion mechanism to enable delivery of a portion of the guidewire into a tracheal cavity of the patient, wherein the insertion mechanism comprises at least one of:
(i) a needle; and
(ii) a sheath.

7. An intubation system as recited in claim 6, wherein the insertion mechanism is configured to receive the guidewire at an insertion channel that allows the guidewire to selectively pass therethrough.

8. An endotracheal intubation kit comprising:
a guidewire; and
a coupling mechanism configured to selectively couple the guidewire to a standard endotracheal tube for the intubation of the endotracheal tube into a patient, wherein the coupling mechanism comprises at least one of:
(i) a clamp; and
(ii) an inflatable bulb.

9. An endotracheal intubation kit as recited in claim 8, further comprising an insertion mechanism configured to facilitate insertion of the guidewire into the patient, wherein the insertion mechanism includes an insertion channel that is configured to receive the guidewire.

10. An endotracheal intubation kit as recited in claim 9, wherein the insertion mechanism comprises a sheath that is configured to be inserted into the patient.

11. An endotracheal intubation kit as recited in claim 9, wherein the inflatable bulb is coupled to the guidewire and is configured to pass through the insertion channel to enable delivery of the guidewire to a tracheal cavity of a patient when the bulb is deflated, and wherein the bulb is configured to couple the guidewire to at least a portion of the endotracheal tube when the bulb is inflated.

12. An endotracheal intubation kit as recited in claim 10, further comprising a pump to selectively inflate the bulb.

13. An endotracheal intubation kit as recited in claim 10, further comprising the endotracheal tube having a channel that extends from a proximal end of the tube to a distal end of the tube, and a hub that is selectively coupled to the distal end of the tube.

14. An endotracheal intubation kit as recited in claim 8, wherein the guidewire includes a coating on at least a portion thereof.

15. A method for performing endotracheal intubation, the method comprising the steps for:
threading a guidewire from a portion of a tracheal cavity of a patient through an airway of the patient;
coupling the guidewire to a standard endotracheal tube;
using the guidewire to intubate the standard endotracheal tube into the patient; and
removing the guidewire from the patient.

16. A method as recited in claim 15, wherein the step for using comprises the step for withdrawing the guidewire down the airway to the tracheal cavity to position a distal end of the tube in the tracheal cavity.

17. A method as recited in claim 16, further comprising the step for decoupling the guidewire from the tube.

18. A method as recited in claim 17, wherein the step for coupling comprises at least one of the steps for:
(i) using a clamp to couple the guidewire to the tube; and
(ii) inflating a bulb coupled to the guidewire to couple the guidewire to the tube.

19. A method as recited in claim 15, further comprising the step for using an insertion mechanism to deliver the guidewire to the tracheal cavity.

20. A method as recited in claim 19, wherein the step for using an insertion mechanism includes the steps for:
placing the insertion mechanism into the patient; and
threading at least a portion of the guidewire through an insertion channel of the insertion mechanism to deliver the guidewire to the portion of the tracheal cavity.
